(19) United States
(12) Patent Application Publication
(10) Pub. No.: US 2006/0025775 A1
(43) Pub. Date: Feb. 2, 2006

(54) Femoral neck resection guide and method

(75) Inventor: Arthur Lalit Laxman Malkani, Prospect, KY (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 10/901,279

(22) Filed: Jul. 28, 2004

(51) Int. Cl. A61F 2/34

(52) U.S. Cl. 606/89

(57) ABSTRACT

A femoral neck resection guide for use in minimally invasive hip surgery is disclosed. The femoral neck resection guide includes at least two spaced resection guide surfaces for guiding a cutting tool and means for attaching the guide surfaces to a portion of the cut section of the neck of the femur. The guide surfaces being spaced apart a distance sufficient to allow resection of the femoral neck.

### FIELD OF THE INVENTION

The present invention relates to orthopedic cutting blocks for use in resecting the neck of a femur, and more particularly, to a femoral neck resection guide for resecting at least a portion of the neck of a femur and facilitating the removal of the same.

### BACKGROUND OF THE INVENTION

At the present time, there is a great emphasis toward minimally invasive techniques in joint replacement surgeries. For example, minimally invasive hip replacements, including two-incision hip arthroplasty and single anterior approach arthroplasty, are currently the preferred methods of performing surgery of this type. While there are benefits to performing these minimally invasive techniques (i.e.—quicker recovery time, less scarring, etc . . . ), there are also requirements and difficulties associated with the methods.

One of the primary objectives during a minimally invasive hip surgical technique is to remove the femoral head and neck portions without dislocating the femoral head from the acetabulum. By not dislocating the femoral head, the hip capsule may be preserved, thereby maintaining stability and power of the hip joint. This is typically accomplished by performing the femoral neck osteotomy and the resection of the femoral head in situ. Unfortunately, there is significant difficulty in performing these resections in situ. The small size of the incisions used in minimally invasive surgery and the lack of instruments directed to performing the bone cuts in such a small space provide significant hurdles for a surgeon.

For the foregoing reasons, there exists a need for a femoral neck resection guide for and method of performing minimally invasive hip resection and bone removal.

### SUMMARY OF THE INVENTION

A first aspect of the present invention is a femoral neck resection guide comprising at least two spaced resection guide surfaces for guiding a cutting tool, the guide surfaces being spaced apart a distance sufficient to allow resection of at least a portion of the femoral neck, and means for attaching the guide surfaces to a portion of the neck of the femur.

Another embodiment of the present invention is a femoral neck resection guide comprising a generally H-shaped body having a top surface and a bottom surface. The body includes two cutting surfaces arranged on opposing sides of the body, the cutting surfaces adapted to make two cuts on a neck section of the femur, at least one aperture for receiving at least one bone connection device to connect the body to the neck section of the femur, and a coupling element adjacent the aperture for engaging an extraction tool while the connection device is engaged to the femoral neck.

Another embodiment of the present invention is a femoral neck resection guide comprising a body. The body has at least two spaced resection guide surfaces for guiding a cutting tool, means for attaching the body and the at least two spaced resection guide surfaces to a portion of the neck of the femur, and a combination insertion and extraction tool form integral with the body.

Another aspect of the present invention is a femoral neck resection instrument system or kit comprising a cutting guide having two spaced resection guide surfaces for guiding a bone resection tool. The cutting guide according to this embodiment includes a bone connection device for connecting the cutting guide to a femoral neck and a tool coupling element. The system or kit also includes an alignment instrument releasably engageable with the cutting guide for aligning the resection guide surfaces and a removal instrument for releasably engaging the tool coupling element of the cutting guide while the bone connection device is connected to the femoral neck.

Another aspect of the present invention is a femoral neck resection guide kit. The kit comprises at least two different sized femoral neck resection guides. Each of the guides includes at least two spaced resection guide surfaces for guiding a cutting tool and means for attaching the guide surfaces to a portion of the neck of the femur. The guide surfaces are spaced apart a distance sufficient to allow resection of substantially all of the femoral neck.

Another aspect of the present invention is a method of removing a neck of a femur comprising providing a femoral neck resection guide having at least two cutting surfaces, aligning the femoral neck resection guide with respect to a femoral neck, connecting the femoral neck resection guide to at least a portion of the neck of the femur, making at least two cuts defining a resection portion, the cuts corresponding to the at least two cutting surfaces of the femoral neck resection guide, and removing the femoral neck resection guide.

### BRIEF DESCRIPTION OF THE DRAWINGS

The present invention will be better understood on reading the following detailed description of non-limiting embodiments thereof, and on examining the accompanying drawings, in which:

FIG. 1 is a top perspective view of the femoral neck resection guide according to an embodiment of the present invention;

FIG. 2 is a bottom plan view of the femoral neck resection guide according to FIG. 1;

FIG. 3 is a front plan view of the femoral neck resection guide according to FIG. 1;

FIG. 4 is right side plan view of the femoral neck resection guide according to FIG. 1;

FIG. 5 is a front plan view of the femoral neck resection guide according to another embodiment of the present invention;

FIG. 6 is a top perspective view of the femoral neck resection guide according to FIG. 1 with an alignment guide adjacent thereto;

FIG. 7 is a top perspective view of the femoral neck resection guide according to FIG. 1 placed adjacent the neck of the femur, with an alignment guide attached thereto and a screw adjacent thereto;

FIG. 8 is a top perspective view of the femoral neck resection guide according to FIG. 1 attached to the neck of the femur;

FIG. 9 is a top perspective view of the femoral neck resection guide according to FIG. 1 attached to the neck of the femur with a saw blade engaged therein;

FIG. 10 is a top perspective view of the femoral neck resection guide according to FIG. 1 attached to the neck of the femur with a saw blade engaged therein in an angled fashion;

FIG. 11 is a top perspective view of the femoral neck resection guide according to FIG. 1 with an extraction tool adjacent thereto;

FIGS. 12a-12c shows a sequence of attaching an extraction tool of FIG. 11 to the femoral neck resection guide; and

FIG. 13 is a top perspective view of the femoral neck resection guide being removed by the extraction tool along with a portion of the femoral neck subsequent to the cutting of the femoral neck.

### DETAILED DESCRIPTION

In describing the preferred embodiments of the subject matter illustrated and to be described with respect to the drawings, specific terminology will be resorted to for the sake of clarity. However, the invention is not intended to be limited to the specific term and includes all technical equivalence which operates in a similar manner to accomplish a similar purpose.

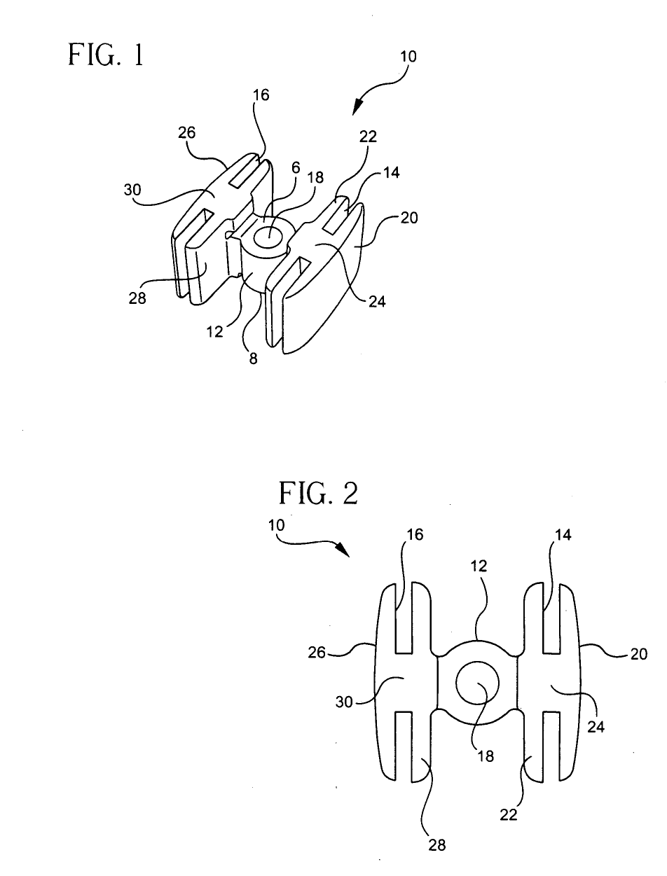

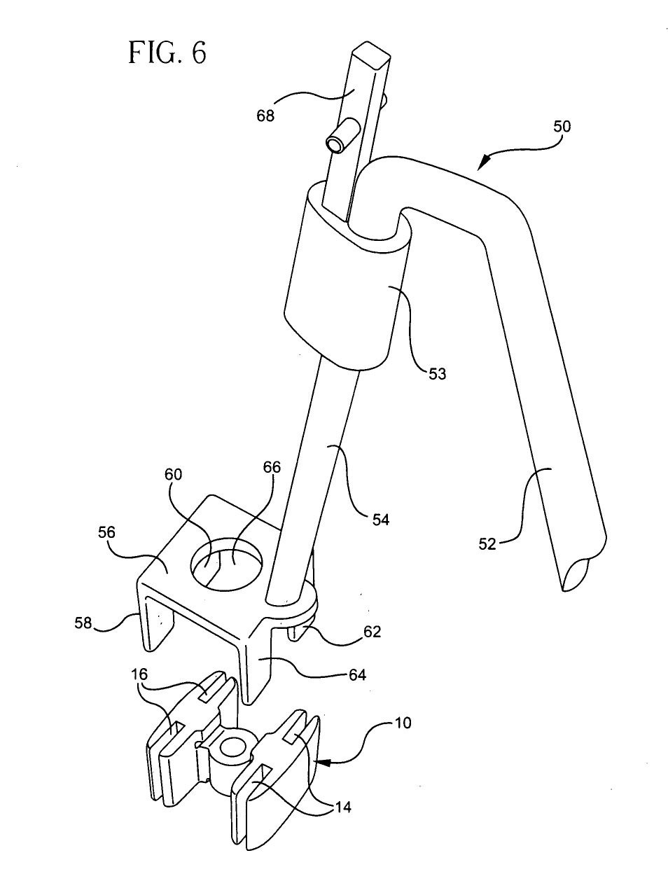

Referring to the drawings, wherein like reference numerals represent like elements, there is shown in the Figures, in accordance with embodiments of the present invention, a femoral neck resection guide designated generally by reference numeral 10. In a preferred embodiment, as shown in the FIGS. 1-4, resection guide 10 is designed to be used in resecting the femoral neck. However, it is contemplated that other embodiments of the present invention can be designed to be used in resecting other bones in other areas of the body. As shown in FIG. 1, resection guide 10 is of unitary construction having a top surface 6 and a bottom surface 8. Resection guide 10 is adapted for attaching to a neck portion 2 of a femur 1, with bottom surface 8 contacting the bone surface. In a preferred embodiment, resection guide 10 includes a body 12, first cutting slot 14, second cutting slot 16, and aperture 18 for facilitating connection to the aforementioned neck portion 2 of femur 1. It should be noted that in preferred embodiments, slots 14 and 16 are spaced so that substantially all of the femoral neck may be resected. However, it is contemplated that slots 14 and 16 can be spaced and distance capable to provide a desired resected portion.

As shown in FIGS. 1-4, body 12 is of a generally cylindrical shape with aperture 18 extending through its center and cutting slots 14 and 16 attached on either side. It is contemplated that body 12 can be of any shape or size, can be configured so as to include any number of apertures 18 at any location and can be attached to any number of cutting surfaces in any manner suitable for facilitating the cutting of a bone. For example, body 12 can be square shaped and include aperture 18 at any portion thereon and cutting surfaces 14 and 16 formed along any side of the square. A rounded shape helps prevent soft tissue damage from occurring during insertion and removal.

As shown in FIGS. 1-4, slots 14 and 16 define cutting surfaces for guiding a cutting instrument. These slots are substantially identical and are attached to body 12 on opposing sides. However, it is contemplated that cutting slots 14 and 16 can be of different configurations and can be attached to body 12 in any manner necessary to facilitate the cutting of a particular bone. In the preferred embodiment shown in the Figures, cutting slots 14 and 16 are formed integral with body 12. However, it is contemplated that pieces including cutting slots 14 and 16 can be formed separately and thereafter permanently or detachably connected to body 12. In the case where the pieces are detachably connected, resection guide 10 is of a modular design, allowing for assembly prior to being inserted into the body or in situ. Assembling in situ can be useful in inserting pieces of resection guide 10 through very small incisions.

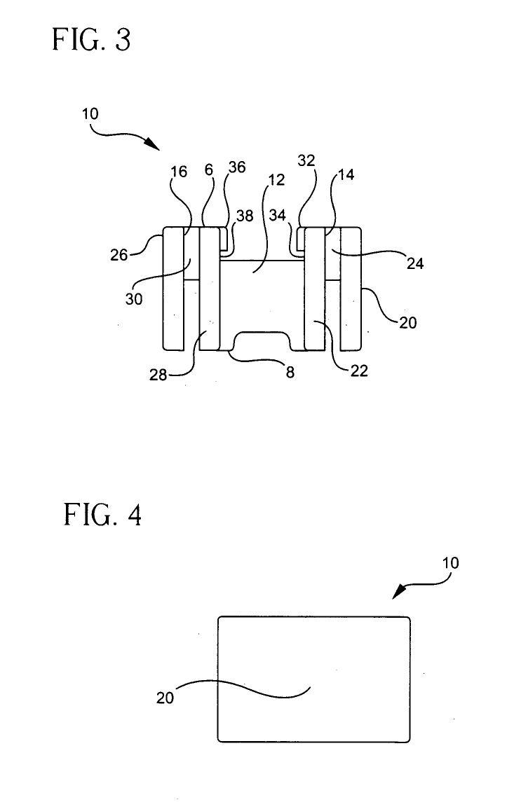

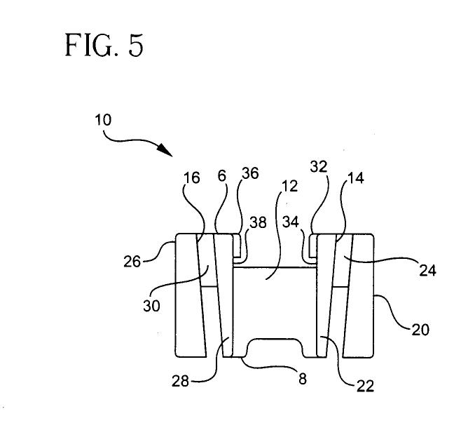

In the preferred embodiment, first cutting slot 14 is formed by first exterior wall 20, first interior wall 22, connected by first bridge 24. Second cutting slot 16 is formed by second exterior wall 26, second interior wall 28, connected by second bridge 30. First exterior wall 20 and first interior wall 22 are connected together by first bridge 24. Bridge 24 is narrower and shorter in height (from the top surface 6 towards the bottom surface 8) than first exterior wall 20 and first interior wall 22, which are substantially the same in dimension and shape. Essentially, bridge 24 extends between first exterior wall 20 and first interior wall 22, to create the slot defined by the two walls. Bridge 24 only extends partially on the depth of slot 14 from top surface 6 towards bottom surface 8 (best shown in FIG. 3) and only extends partially along the depth of first exterior wall 20 and first interior wall 22 (best shown in FIG. 2). At the connection of first cutting slot 14 and body 12, a ledge 32 is formed. This ledge 32 hangs over body 12 to form a groove 34 for aiding in the connection with other tools. This will be discussed further below. In a substantially similar fashion, second exterior wall 26 and second interior wall 28 are connected together by second bridge 30, and second cutting slot 16 forms a ledge 36 and groove 38. Since the bottom of slots 14 and 16 are left open, this allows greater travel of the saw blade along the guide slot.

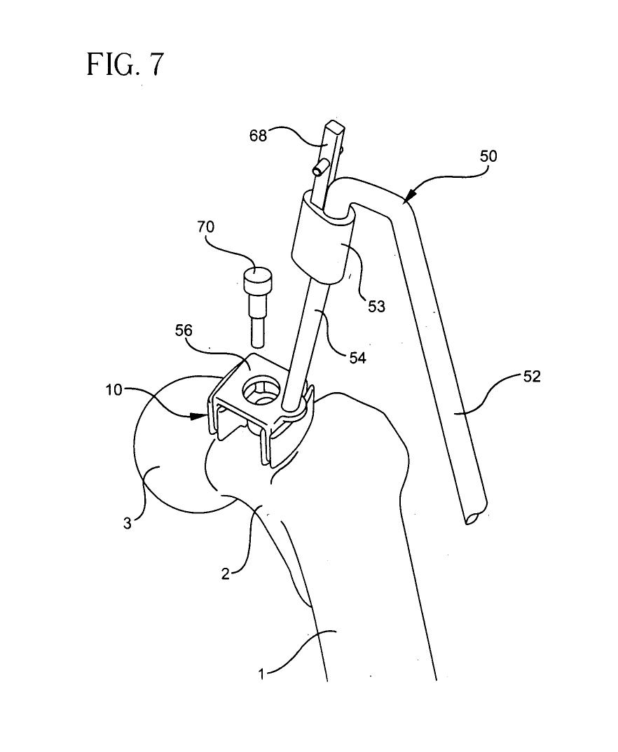

As shown in FIG. 5, cutting slots 14 and 16 may be non-parallel or angled inwardly from top surface 6 to bottom surface 8, at an angle A. This angle allows for cuts to made at a corresponding angle, thereby creating a cut section of bone that is likewise angled inwardly from a top surface to a bottom surface. Such a configuration is desirable for facilitating the easy removal of both the guide and the cut section of bone from the body of a patient. In certain embodiments, angle A is approximately five degrees. However, it is contemplated that angle A can be any amount for creating a desired cut section of bone.

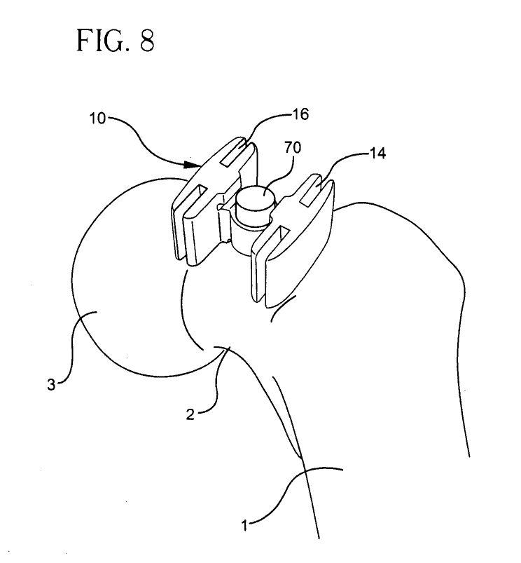

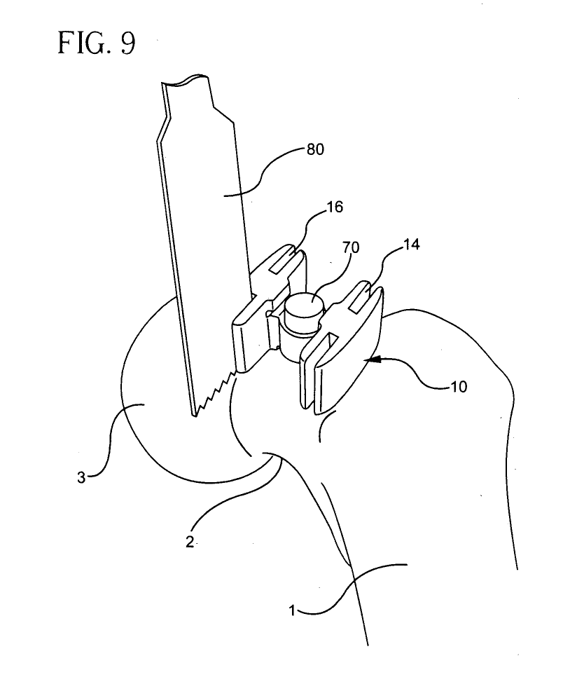

FIGS. 6 and 7 illustrate an alignment guide, generally denoted as 50, and its cooperation with resection guide 10. As shown in FIGS. 6 and 7, alignment guide 50 includes elongate guide portion 52 having one end coupled to a coupler 53. A shaft 54 extends from coupler 53 and includes a platform 56 having four fingers 58, 60, 62, and 64, and central opening 66. Furthermore, alignment guide 50 may optionally include navigation tracker mount 68 for utilizing computer or other navigation tracking systems. The use of such a tracker is shown in U.S. Pat. Nos. 6,021,343 and 6,434,415, the disclosures of which are herby incorporated by reference herein. Elongate guide portion 52 acts as a handle to easily manipulate alignment guide 50. Coupler 53 provides a connection point for both shaft 54 and navigation tracker mount 68. Since shaft 54 connects with platform 56 manipulation of elongate portion 52 will, in turn, manipulate platform 56. Fingers 58, 60, 62, and 64 extend from platform 56 and are adapted to mate with slots 14 and 16 of resection guide 10. Opening 66 is essentially a hole through platform 56, allowing for access to body 12 of resection guide 10, when alignment guide 50 is engaged with resection guide 10.

In operation, as shown in FIG. 7, fingers 58, 60, 62, and 64 of alignment guide 50 are received within cutting slots 16 and 14 of resection guide 10. Fingers 58 and 60 extend into cutting slot 16 on either side of second bridge 30, while fingers 62 and 64 extend into cutting slot 14 on either side of first bridge 24. The fit between the finger and the slots is such that, absent a force, resection guide 10 remains engaged with alignment guide 50. However, it is contemplated that other coupling methods can be employed for connecting alignment guide 50 to resection guide 10 such as spring loaded quick releases or ball detents. Nevertheless, these mating relationships between resection guide 10 and alignment guide 50 allow for a surgeon to direct resection guide 10 through small incisions and into contact with a bone surface, such as neck portion 2 of femur 1, as shown in FIG. 7. If the resection guide 10 is modular and assembled in situ, guide 50 can engage slots 14 and 16 after being inserted through the incision.

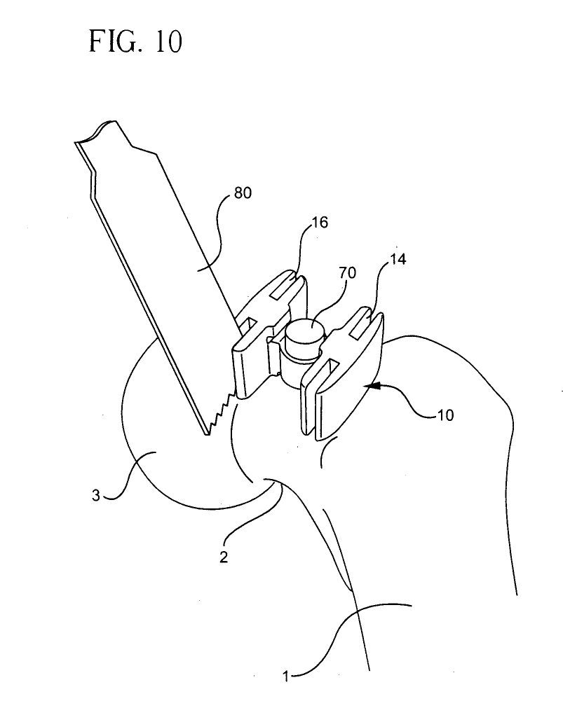

In an embodiment of the present invention, guide 10 is aligned so that slot 14 is positioned to allow for a cut which matches the angle of the femoral component as it would rest on the calcar (shown in FIGS. 7 and 8). This would allow a surgeon to simply utilize slot 14 to create the final cut for which the subsequently inserted femoral component would rest against. In other embodiments, guide 10 is aligned so that at least a portion of neck portion 2 of femur 1 can be resected. It is contemplated that the portion which is resected can be any size and can be oriented in any manner which allows for at least a portion of the femoral neck 2 to be removed. In other words, guide 10 can be aligned so that slots 14 and 16 allow for cuts to be made at any angle with respect to femur 1, as long as a portion of the neck is removed. Any removal of a portion of neck 2 necessarily detaches femoral head 3 from femur 1, thereby allowing for removal of head 3. If one of the cuts does not match the angle of the femoral component as it would rest against the calcar, at least one subsequent cut would need to be made to match the angle. It is contemplated that guide 10 can be aligned by various means. For example, alignment guide 50 may be configured so that shaft 52 may be aligned with the axis of femur 1. In this embodiment, aligning shaft 52 with the axis of femur 1 would automatically align guide 10 in a correct orientation. Similarly, as mentioned above, a navigation tracker may be utilized in order to properly align resection guide 10.

As is shown in FIG. 7, a screw 70 is provided for connecting resection guide 10 with a bone surface. Screw 70 has a head and a threaded portion and may be a standard bone screw known to one of ordinary skill in the art. It is contemplated the other means for attaching resection guide 10 to the bone. For example, a surgeon can utilize pins, nails, adhesive, among others, to attach resection guide 10 to a bone surface. Furthermore, it is also contemplated to utilize more than one attachment means for connecting resection guide 10 to a bone. For example, in another embodiment, resection guide 10 can be connected to a bone surface by two or more screws or two or more bone pins.

In operation, as best shown in FIG. 7, screw 70 is inserted through opening 66 of alignment guide 50 and into aperture 18 of resection guide 10. Screw 70 is then screwed into the bone using a typical tool such as a screw driver or drill until the head resets on the top surface of body 12. However, it is contemplated that prior to inserting screw 70 into aperture 18, opening 66 and aperture 18 can guide a drill or other hole forming tool to pre-form a hole. Thereafter, screw 70 can more easily be screwed into the bone. In a preferred embodiment, shown in FIG. 6, screw 70 is a self-tapping screw capable of creating a hole absent a drill or other hole making tool. Once the head of screw 70 is in engagement with resection guide 10, and in the preferred embodiment shown in the Figures, neck portion 2 of femur 1, alignment guide 50 can be removed from resection guide 10. This accomplished by disengaging fingers 58, 60, 62, and 64 from slots 14 and 16.

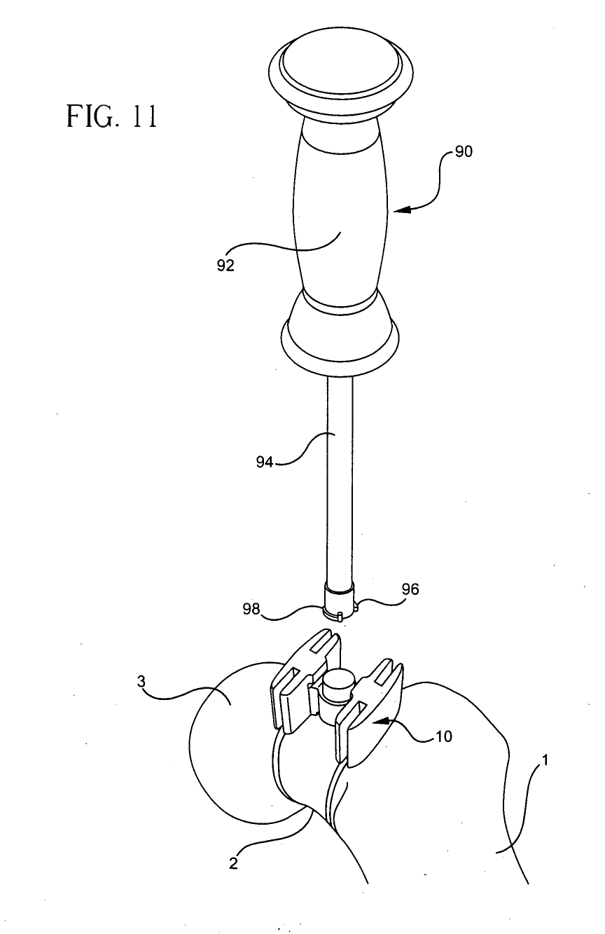

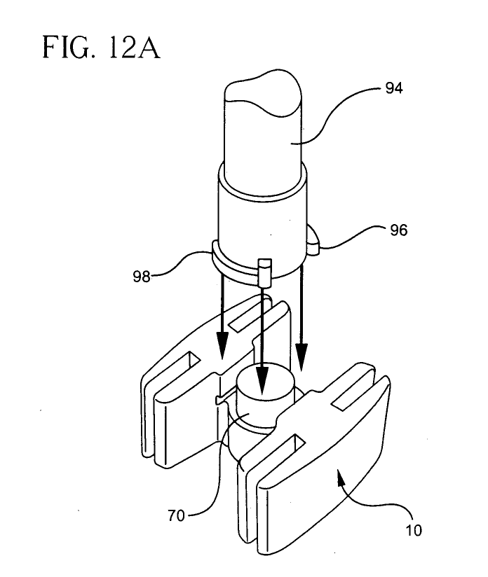

Upon removal of alignment guide 50, as shown in FIG. 8, resection guide 10 is attached to neck portion 2 of femur 1, in a position ready for a cutting operation. As shown in the figure, cutting slots 14 and 16 are aligned over the proximal and distal ends of neck portion 2. This allows for a resected neck portion 4, created using resection guide 10, to consist of the majority of neck portion 2. While use of guide 10 with respect to the femur is described, resection guide 10 can be aligned to cut any section of bone, in any part of the body. During the cutting process, a saw blade 80 is inserted into each slot 14 and 16, and the slot is used to guide saw blade 80 along and through the bone, as shown in FIG. 9. In operation, the surgeon inserts saw blade 80 into one slot and into contact with bone. The surgeon then moves the blade to cut and separate a cut portion 4 from the remainder of the bone. This includes angling saw blade 80 to navigate the saw around bridges 24 and 30, as shown in FIG. 10. It is contemplated that any suitable type of cutting tool can be utilized to perform these steps. For example, resection guide 10 can be used in conjunction with a milling device.

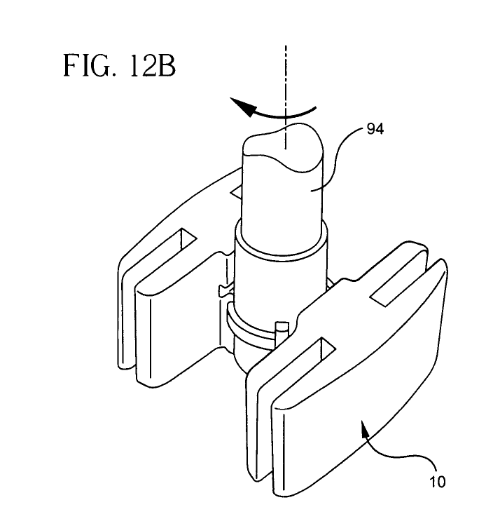

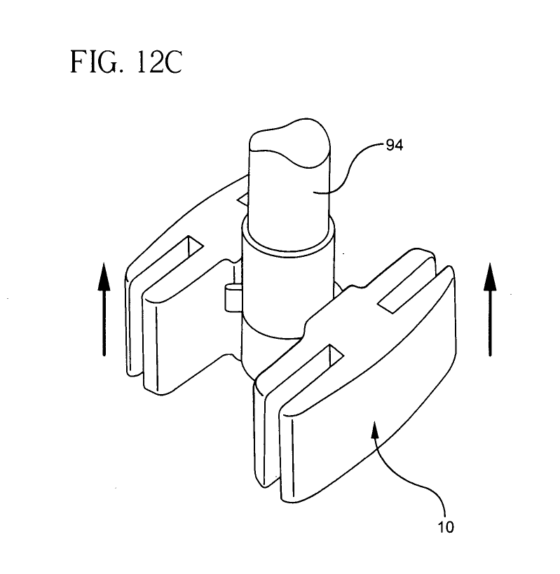

FIG. 11 depicts an extraction tool, generally denoted as 90. This tool includes a handle 92, at one end thereof, a shaft 94, and a coupling 96, at the other end thereof. Handle 92 is dimensioned for grasping by a surgeon. In the preferred embodiment, shaft 94 is generally cylindrical and connects handle 92 to coupling 96. Coupling 96 is configured so as to cooperate and preferably rotatably connect with resection guide 10. In a preferred embodiment, as shown in FIGS. 10-12, coupling 96 is configured for insertion into grooves 34 and 38 of resection guide 10. Coupling 96 is substantially cylindrical and includes two radially extending male portions 98 for reception under ledges 32 and 36 in grooves 34 and 38 on resection guide 10. Initially, coupling 96 is brought adjacent to body 12 without male portions 98 being in contact with grooves 34 and 38, however upon a clockwise turning of handle 90, and thus coupling 96, male portions 98 become disposed under ledges 32 and 36 and engage grooves 34 and 38 portions. This results in extraction tool 90 being fixably attached to resection guide 10. At this point, any movement applied to extraction tool 90 also moves resection guide 10. This is best shown in the sequence depicted in FIGS. 12a-12c. It is contemplated that extraction tool 90 can be configured and dimensioned in different ways. Similarly, coupling 96 can cooperate with resection guide 10 in many different fashions. For example, in other embodiments, coupling 96 can snap into a corresponding portion of resection guide 10 or screw into a threaded portion of resection guide 10.

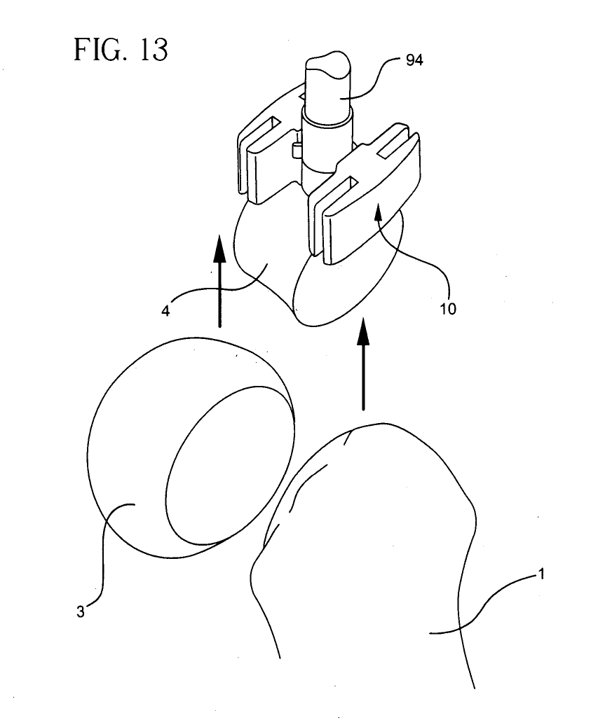

The removal of the created resected portion 4 of neck portion 2 of femur 1 is shown in FIG. 13. With the aforementioned extraction tool 90 connected to resection guide 10, the surgeon, subsequent to the cuts being completed on the bone, removes resection guide 10 while it is connected to resected portion 4 by screw 70. Typically, resected portion 4 will be small enough to fit through the incision created in the tissue of a patient in order to insert resection guide 10. Furthermore, it should be noted that the exterior walls 20 and 26 of slots 14 and 16 may be curved to minimize the amount of soft tissue damage created by inserting and removing resection guide 10. However, it is foreseeable that any incisions can be stretched utilizing retractors or the like or resected portion 4 can be further cut into smaller pieces in situ. Upon removal of resected portion 4, the now resected head 3 remains in the acetabulum of the patient. Thereafter, head 3 can be resected or morsalized to allow for its removal through the incision. This can be done in any fashion known to one of ordinary skill in the art.

It is contemplated that in other embodiments of the present invention, a device like alignment handle 52 or extraction tool 90 can be formed integral with resection guide 10. In these embodiments, extraction tool 90 would be operable for both insertion/alignment and removal of resection guide 10. It should be noted that an extraction tool of this type would need to be configured so as to allow for resection of the bone while being coupled with a resection guide. Furthermore, an extraction tool for use in these embodiments of the present invention would also need to allow for the connection of resection guide 10 to the bone. In certain of these embodiments, the extraction tool could be configured to allow for a screw to be inserted into a hole in resection guide 10 that is located away from the connection between the extraction tool and the guide. However, it is also contemplated that the extraction tool can be formed to include a cannula-like tube that a screw can be inserted through. In this embodiment, the screw would engage a surface, not unlike in the preferred embodiment shown in the Figures, and fixably attach resection guide 10 and extraction tool 90 to the bone.

Another aspect of the present invention is a method for resecting and removing the neck portion of a femur. The method according to this aspect of the invention includes the step of providing a femoral neck resection guide 10 as discussed above. The femoral neck resection guide 10 provided can be in accordance with any of the various embodiments discussed above. Resection guide 10 is then connected to the portion of the femoral neck which is to be cut. It is contemplated that resection guide 10 can be configured for and attached to other bones in accordance with the present invention. However, in a preferred embodiment as discussed in this description of a preferred method in accordance with the present invention, the bone to be resected is the neck portion 2 of femur 1. As discussed above, the connection of resection guide 10 to neck portion 2 can be accomplished in multiple fashions (i.e.—with screws, pins, nails, etc . . . ). In a preferred embodiment, resection guide 10 is connected to neck portion 2 by a self tapping screw 70.

In the preferred method of use, a surgeon attaches alignment guide 50 to resection guide 10. The surgeon then utilizes alignment guide 50 to insert resection guide 10 into and through a previously created incision in the tissue of a patient. It is noted that this previously created incision can be of any size in any portion of the body. For purposes of discussing a preferred method according to an aspect of the invention, the incision lies in the hip region of a patient and is of a generally small size of approximately 4-6 centimeters (i.e.—minimally invasive). Once resection guide 10 is inserted into the incision, it is brought into contact with neck portion 2 of femur 1. As mentioned above, it is contemplated that alignment guide 50 can be used in conjunction with a navigation tracker mount 68, which provides an electronic mode of navigating resection guide 10 into place. Alternatively, as is also mentioned above, guide 10 can be oriented using guide 50 by aligning portion 52 with the mechanical axis of the femur. Once resection guide 10 is properly positioned, screw 70 is inserted into the incision and into contact with resection guide 10 at aperture 18. The surgeon then threads or advances screw 70 into the bone material of neck portion 2, utilizing any tool useful in performing the function (e.g. a screwdriver or drill). Once resection guide 10 is properly attached to neck portion 2, alignment guide 50 can removed therefrom.

With resection guide 10 in place, the surgeon may now resect the bone to create a resected portion 4. In a preferred embodiment, a surgeon utilizes an oscillating saw blade 80 make to cuts through the bone that correspond to cutting surfaces 14 and 16, respectively. It is contemplated that other cutting devices, such as a reciprocating saw, can also be utilized. As mentioned above, saw blade 80 must be manipulated in a manner to completely resect the bone and create resected portion 4. This requires a surgeon to angle saw blade 80 around bridges 24 and 30 extending between slots 14 and 16. Upon completion of the cuts and creation of cut portion 4, saw blade 80 is removed from the incision.

At this point in the surgical procedure, cut portion 4 is separated from the main portion of femur 1 and femoral head 3 (best shown in FIG. 13). However, resection guide 10 is still connected to resected portion 4, the result of opposing cuts being made on the bone. The next step relates to removing the resection guide 10 and resected portion 4 together. While there are many devices that can be used to remove resection guide 10 from the body, a preferred method in accordance with the present invention uses an extraction tool 90. In use, a surgeon inserts extraction tool 90 into the incision and moves coupling 96 adjacent to resection guide 10. The surgeon can grasp and guide extraction tool 90 by holding handle 92. The surgeon then connects male portions 98 located on coupling 96 with grooves 34 and 38 located on resection guide 10, by rotating extraction tool 90 to lock the two instruments together. Now, any movement imparted upon extraction tool 90 will likewise be imparted on resection guide 10. The surgeon then simply removes resection guide 10, along with resected portion 4 of femur 1, through the incision. Thereafter, femoral head 3 can be resected in situ, using known methods, or removed through the incision, if the incision size permits. The surgeon may now perform the remaining steps in the surgery to be performed (e.g.—total hip replacement surgery).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A femoral neck resection guide comprising:
at least two spaced resection guide surfaces for guiding a cutting tool, said guide surfaces being spaced apart a distance sufficient to allow resection of at least a portion of the femoral neck; and
means for attaching said guide surfaces to a portion of the neck of the femur.

2. The femoral neck resection guide according to claim 1, wherein said guide surfaces are non-parallel.

3. The femoral neck resection guide according to claim 1, further comprising coupling means for releasably coupling an insertion and removal tool to said body.

4. The femoral neck resection guide according to claim 1, wherein the means for attaching said guide surfaces is a screw inserted into said body at a point intermediate to said spaced guide surfaces.

5. The femoral neck resection guide according to claim 1, wherein said guide surfaces are connected to a body.

6. The femoral neck resection guide according to claim 5, wherein said guide surfaces are formed integral with the body.

7. The femoral neck resection guide according to claim 1, wherein said guide surfaces are slots.

8. The femoral neck resection guide according to claim 1, further including an alignment guide to position said femoral neck resection guide.

9. The femoral neck resection guide according to claim 8, wherein the alignment guide includes a navigation tracker mount for connecting to a navigation tracker to aid in aligning said femoral neck resection guide.

10. The femoral neck resection guide according to claim 1, wherein the means for attaching said guide surfaces is a pin inserted at a point intermediate to said spaced guide surfaces.

11. The femoral neck resection guide according to claim 1, wherein the means for attaching said guide surface is a nail inserted at a point intermediate to said spaced guide surfaces.

12. The femoral neck resection guide according to claim 1, wherein said femoral neck resection guide includes two spaced resection guide surfaces.

13. The femoral neck resection guide according to claim 12, wherein the two spaced resection guide surfaces are opposed.

14. The femoral neck resection guide according to claim 1, wherein said guide surfaces are spaced apart a distance sufficient to allow resection of substantially all of the femoral neck.

15. A method of removing a neck of a femur comprising:
providing a femoral neck resection guide having at least two cutting surfaces;
aligning the femoral neck resection guide with respect to a femoral neck;
connecting the femoral neck resection guide to at least a portion of the neck of the femur;
making at least two cuts defining a resected portion, the cuts corresponding to the at least two cutting surfaces of the femoral neck resection guide; and
removing the femoral neck resection guide.

16. The method according to claim 15, wherein removal of the femoral neck resection guide simultaneously facilitates removal of the resected portion of the femur.

17. The method according to claim 15, wherein the alignment step further includes the step of providing an alignment guide for connection to the femoral neck resection guide.

18. The method according to claim 17, wherein the step of aligning the femoral resection guide using the alignment guide is performed prior to connecting the femoral resection guide to at least a portion of the neck of the femur.

19. The method according to claim 18, further including the step of removing the alignment guide after connecting the femoral resection guide to at least a portion of the neck of the femur.

20. The method according to claim 15, wherein said step of making at least two cuts includes utilizing a saw adapted to cut bone.

21. The method according to claim 20, wherein the at least two cutting surfaces are slots for facilitating cutting by the saw.

22. The method according to claim 15, wherein the step of connecting the femoral neck resection guide to the at least a portion of the neck of the femur further includes screwing a screw into the neck portion.

23. The method according to claim 15, wherein the step of connecting the femoral neck resection guide to at least a portion of the neck of the femur further includes connecting a pin to the neck portion.

24. The method according to claim 15, wherein the step of connecting the femoral neck resection guide to at least a portion of the neck of the femur further includes connecting a nail to the neck portion.

25. The method according to claim 15, wherein alignment step further includes utilizing a navigation tracker to position the femoral neck resection guide on the neck portion of the femur.

26. The method according to claim 15, further including the step of removing a head of the femur.

27. The method according to claim 15, further including the step of providing an extraction tool for use in extracting the resected portion and femoral neck resection guide.

28. The method according to claim 27, further including the step of connecting the extraction tool to the femoral neck resection guide.

29. The method according to claim 28, wherein said step of removing the femoral neck resection guide is performed by utilizing the extraction tool.

30. The method according to claim 15, wherein said step of making at least two cuts includes making at least two non-parallel cuts.

31. A femoral neck resection guide comprising:
a generally H-shaped body having a top surface and a bottom surface, the body including;
two cutting surfaces arranged on opposing sides of said body, the cutting surfaces adapted to make two cuts on the neck section of the femur;
at least one aperture for receiving at least one bone connection device to connect said body to a neck section of the femur; and
a coupling element adjacent said aperture for engaging an extraction tool while said connection device is engaged to the femoral neck.

32. The femoral neck resection guide according to claim 31, wherein said two cutting surfaces are non-parallel.

33. The femoral neck resection guide according to claim 31, wherein the bone connection device is a screw.

34. The femoral neck resection guide according to claim 31, wherein the bone connection device is a pin.

35. The femoral neck resection guide according to claim 31, wherein the bone connection device is a nail.

36. The femoral neck resection guide according to claim 31, wherein the cutting surfaces are slots.

37. The femoral neck resection guide according to claim 31, said body including two apertures for receiving two bone connection devices.

38. The femoral neck resection guide according to claim 31, further including a removable alignment guide for manipulating said femoral resection guide.

39. The femoral neck resection guide according to claim 38, wherein the alignment guide includes a navigation tracker mount.

40. A femoral neck resection instrument system or kit comprising:
a cutting guide having two spaced resection guide surfaces for guiding a bone resection tool, said cutting guide including a bone connection device for connecting said cutting guide to a femoral neck and a tool coupling element;
an alignment instrument releasably engageable with said cutting guide for aligning said resection guide surfaces; and
a removal instrument for releasably engaging the tool coupling element of said cutting guide while said bone connection device is connected to the femoral neck.

41. The femoral neck resection instrument system or kit of claim 40, wherein the two spaced resection guide surfaces are non-parallel.

42. The femoral neck resection instrument system or kit of claim 40, wherein the bone connection device is a screw.

43. The femoral neck resection instrument system or kit of claim 40, wherein said alignment instrument includes a navigation tracker to aid in aligning said cutting guide.

44. The femoral neck resection instrument system or kit of claim 40, wherein the cutting guide surfaces are parallel.

45. A femoral neck resection guide kit comprising:
at least two different sized femoral neck resection guides, each of said guides including at least two spaced resection guide surfaces for guiding a cutting tool, said guide surfaces being spaced apart a distance sufficient to allow resection of substantially all of the femoral neck, and means for attaching said guide surfaces to a portion of the neck of the femur.

46. The femoral neck resection guide kit according to claim 45, further comprising an alignment guide to position said guide.

47. The femoral neck resection guide kit according to claim 45, further comprising a removal instrument for removing said guide.

48. A femoral neck resection guide comprising:
a body having at least two spaced resection guide surfaces for guiding a cutting tool;
means for attaching said body and the at least two spaced resection guide surfaces to a portion of the neck of the femur; and
a combination insertion and extraction tool formed integral with said body.
